(19) United States
(12) Patent Application Publication
(10) Pub. No.: US 2026/0314907 A1
(43) Pub. Date: Oct. 15, 2026

(54) ENDOSCOPE PROCESSOR AND ENDOSCOPE SYSTEM

(71) Applicant: OLYMPUS MEDICAL SYSTEMS CORP., Tokyo (JP)

(72) Inventors: Daiki ARIYOSHI, Tokyo (JP); Shinsuke HOMMA, Tokyo (JP)

(73) Assignee: OLYMPUS MEDICAL SYSTEMS CORP., Tokyo (JP)

(21) Appl. No.: 19/690,549

(22) Filed: May 28, 2026

Related U.S. Application Data
(63) Continuation of application No. PCT/JP2024/039252, filed on Nov. 5, 2024.

(30) Foreign Application Priority Data
Nov. 29, 2023 (JP) 2023-201871

(51) Int. Cl. A61B 1/00; A61B 5/00; A61B 5/103; G06T 7/00; G06T 7/90; G06T 11/10; H04N 23/88

(52) U.S. Cl. CPC A61B 1/00009; A61B 1/0005; A61B 5/1032; A61B 5/4842; A61B 5/746; G06T 7/0016; G06T 7/90; G06T 11/10; G06T 2207/10024; G06T 2210/62; H04N 23/88

(57) ABSTRACT

An endoscope processor includes a connection interface and processing circuitry. The processing circuitry is configured to receive, via the connection interface, an image signal to generate an object image, normalize, by a blue pixel value, a sum of a red pixel value and a green pixel value of at least one pixel of the object image, thereby calculating an index of the at least one pixel, identify, based on the index, which of a plurality of symptom levels defined using a plurality of thresholds the at least one pixel falls into, acquire an identification color corresponding to the identified symptom level, and generate an identification image using the identification color of the at least one pixel.

### CROSS REFERENCE TO RELATED APPLICATION

This application is a continuation of International Application No. PCT/JP2024/039252, filed on Nov. 5, 2024, which claims priority to Japanese Patent Application No. 2023-201871, filed on Nov. 29, 2023, the contents of which are incorporated herein by reference in its entirety.

### FIELD

The present disclosure relates to an endoscope processor and an endoscope system including an endoscope for picking up an image of an object in a body.

### BACKGROUND

International Publication No. 2018/230130 discloses an endoscope apparatus that calculates an index indicating a degree of abnormality of an object in accordance with a color included in an object image captured by an endoscope, and identifies and displays the index according to a threshold.

### SUMMARY

According to aspects of the present disclosure, an endoscope processor is provided, which includes a connection interface and processing circuitry. The processing circuitry is configured to receive, via the connection interface, an image signal to generate an object image. The processing circuitry is further configured to normalize, by a blue pixel value, a sum of a red pixel value and a green pixel value of at least one pixel of the object image, thereby calculating an index of the at least one pixel. The processing circuitry is further configured to, based on the index, identify which of a plurality of symptom levels defined using a plurality of thresholds the at least one pixel falls into. The processing circuitry is further configured to acquire an identification color corresponding to the identified symptom level. The processing circuitry is further configured to generate an identification image using the identification color of the at least one pixel.

According to aspects of the present disclosure, further provided is an endoscope system that includes an endoscope processor, an endoscope, a memory, and a monitor. The endoscope processor includes a connection interface and processing circuitry. The processing circuitry is configured to receive, via the connection interface, an image signal to generate an object image. The processing circuitry is further configured to normalize, by a blue pixel value, a sum of a red pixel value and a green pixel value of at least one pixel of the object image, thereby calculating an index of the at least one pixel. The processing circuitry is further configured to, based on the index, identify which of a plurality of symptom levels defined using a plurality of thresholds the at least one pixel falls into. The processing circuitry is further configured to acquire an identification color corresponding to the identified symptom level. The processing circuitry is further configured to generate an identification image using the identification color of the at least one pixel. The endoscope is connected to the endoscope processor via the connection interface. The endoscope is configured to output the image signal. The memory is connected to the endoscope processor. The memory is configured to store the thresholds and identification color data. The monitor is connected to the endoscope processor.

### BRIEF DESCRIPTION OF DRAWINGS

FIG. 1 is a diagram showing a configuration of an endoscope apparatus according to an embodiment of the present disclosure.

FIG. 2 is a diagram for illustrating a relationship between optical absorption characteristics of plasma and optical emission characteristics of a light source.

FIG. 3 is a diagram for illustrating optical absorption characteristics and the like of a subject.

FIG. 4 is a diagram for illustrating calculation formulas for an index in the endoscope apparatus according to the embodiment of the present disclosure.

FIG. 5 is a diagram for illustrating calculation formulas for an index in the endoscope apparatus according to the embodiment of the present disclosure.

FIG. 6 is a flowchart of a method of operating the endoscope apparatus according to the embodiment of the present disclosure.

FIG. 7 is a diagram showing a relationship among symptom levels and thresholds in the endoscope apparatus according to the embodiment of the present disclosure.

FIG. 8 is a diagram showing a relationship among the symptom levels and threshold sets in the endoscope apparatus according to the embodiment of the present disclosure.

FIG. 9 is a diagram showing a relationship among the symptom levels and identification colors in the endoscope apparatus according to the embodiment of the present disclosure.

FIG. 10 is a display example 1 of a screen of a monitor in the endoscope apparatus according to the embodiment of the present disclosure.

FIG. 11 is a display example 2 of the screen of the monitor in the endoscope apparatus according to the embodiment of the present disclosure.

FIG. 12 is a display example 3 of the screen of the monitor in the endoscope apparatus according to the embodiment of the present disclosure.

FIG. 13 is a display example 4 of the screen of the monitor in the endoscope apparatus according to the embodiment of the present disclosure.

FIG. 14 is a display example 5 of the screen of the monitor in the endoscope apparatus according to the embodiment of the present disclosure.

FIG. 15 is a display example 6 of the screen of the monitor in the endoscope apparatus according to the embodiment of the present disclosure.

FIG. 16 is a display example 7 of the screen of the monitor in the endoscope apparatus according to the embodiment of the present disclosure.

### DESCRIPTION OF EMBODIMENTS

### Configuration of Endoscope Apparatus

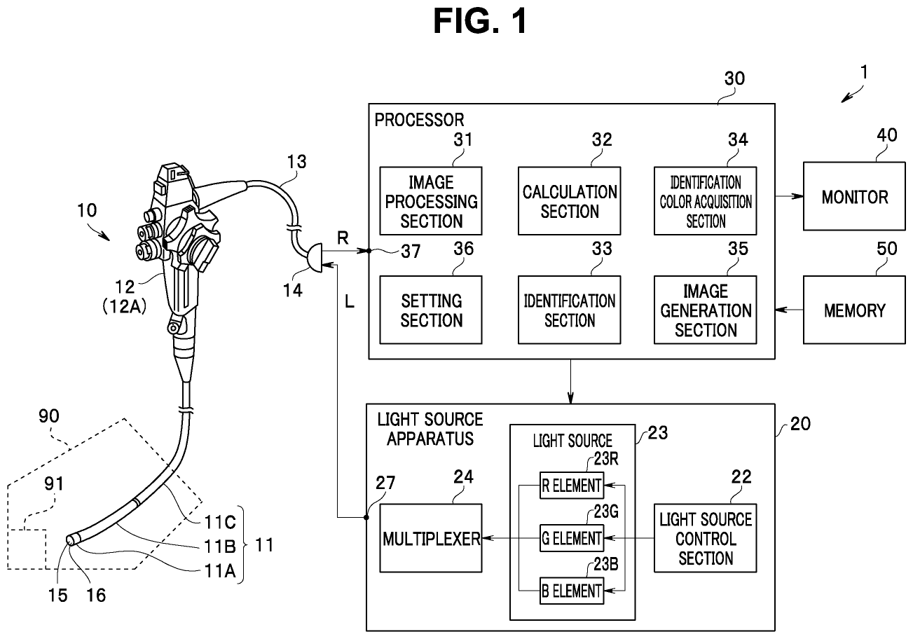

As shown in FIG. 1, an endoscope apparatus 1 according to an embodiment includes an endoscope 10, a light source apparatus 20, a processor 30, a monitor 40, and a memory 50.

The endoscope 10 includes an elongated insertion portion 11 to be inserted into an examinee 90, an operation portion 12 provided at a proximal end of the insertion portion 11, a universal cord 13 extending from the operation portion 12, and a connector 14. The operation portion 12 includes a plurality of buttons 12A and the like, which constitute an endoscope setting section, for operating endoscope functions and image pickup functions. The insertion portion 11 of the endoscope 10 includes, sequentially from a distal end side, a distal end portion 11A, a bending portion 11B provided at a proximal end of the distal end portion 11A, and an elongated flexible tube 11C provided at a proximal end of the bending portion 11B. The distal end portion 11A is provided with an image pickup unit 15, which is an image pickup portion, and an illumination unit 16, which is an illumination portion.

The connector 14 of the endoscope 10 is connected to connection interfaces (see 27, 37 in FIG. 1) of the light source apparatus 20 and the processor 30. Illumination light L produced by the light source apparatus 20 is guided to the illumination unit 16 at the distal end portion 11A of the insertion portion 11, to illuminate the object 91 in the body of the examinee 90. The image pickup unit 15 includes an image pickup device such as a CCD. The image pickup unit 15 converts reflected light R from the object 91 into an electrical signal, thereby outputting an object image to the processor 30.

The light source apparatus 20 includes a light source control section 22, a light source 23, and a multiplexer 24. The light source apparatus 20 further includes the connection interface 27 configured to be connected to the connector 14 of the endoscope 10.

The light source control section 22 is a light source control circuit that is connected to the light source 23 and that controls the light source 23 in accordance with a control signal from the processor 30.

The light source 23 includes a plurality of light-emitting elements, such as LEDs, for example. The light source 23 includes an R element 23R, a G element 23G, and a B element 23B. The R element 23R emits red light Br in the normal band. The G element 23G emits green light Bg in the normal band. The B element 23B emits blue light Bb in the normal band. The B element 23B not only emits blue light Bb in the normal band, but also narrows the bandwidth of the blue light using, for example, a narrow-band light filter (not shown), and outputs narrow-band blue light Nb.

The multiplexer 24 multiplexes a plurality of lights inputted from the light source 23, thereby outputting the illumination light L to the illumination unit 16.

The processor 30 may be implemented by processing circuitry. The processor 30 includes an image processing section 31, a calculation section 32, an identification section 33, an identification color acquisition section 34, an image generation section 35, and a setting section 36. The processor 30 further includes the connection interface 37 configured to be connected to the connector 14 of the endoscope 10. The processor 30, which includes processing circuitry including one or more CPUs, is configured to control the entirety of the endoscope apparatus 1, generate an endoscopic image based on an image pickup signal inputted from the endoscope 10, and generate an identification image based on the endoscopic image, as will be mentioned below.

The setting section 36, which is a setting circuit, is a button or the like with which the user inputs various instructions. The setting section 36 may be a touch panel, a keyboard, a foot switch, a button 12A of the endoscope 10, or the like, which is separate from the processor 30. For example, from the setting section 36, a bending instruction for the bending portion, a drive instruction for the light source apparatus 20, a type of the illumination light L for illuminating the object 91, a type of an observation site of the object 91, an image to be displayed on the monitor 40, and the like are inputted as instructions.

It should be noted that configurations of the image processing section 31, the calculation section 32, the identification section 33, the identification color acquisition section 34, and the image generation section 35 will be described later.

At least one of a plurality of configurations of the processor 30 and the light source control section 22 may be configured by the processor 30 or an internal circuit (CPU) of the light source apparatus, both of which are run by software (program), or may be configured by a dedicated hardware circuit.

The monitor 40 is, for example, an LCD, a CRT, or the like, that displays a color image. The monitor 40 displays an image instructed by the processor 30. The monitor 40 having a touch panel functionality may also configure a part of the setting section 36.

The memory 50 may include at least one of a RAM, a ROM, a hard disk drive device, or the like, storing data such as operating conditions of the processor 30, a program, or the like. The memory 50 may also be an internal memory of the processor 30 where data or the like transferred from a non-transitory computer-readable storage medium such as a CD or a DVD has been stored. The processor 30 performs predetermined processing based on the program and data stored in the memory 50. Past examination data or the like of the examinee 90, which is stored in, for example, a server separate from the endoscope apparatus 1, may be transferred to the memory 50 via an Internet connection or the like.

### Index

The calculation section 32 is a calculation circuit that calculates an index VI of each of a plurality of pixels of an object image outputted by the image pickup unit. The index VI, which quantitatively indicates the symptom level of the object 91, is calculated using a predetermined calculation formula.

The following describes a rationale for selecting the calculation formula.

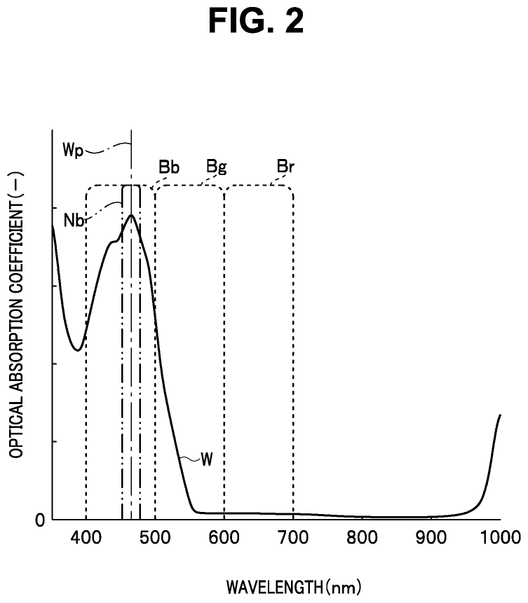

FIG. 2 is a diagram for illustrating a relationship between optical absorption characteristics W of plasma and wavelength of light produced by the light source 23. FIG. 2 shows red light Br in the normal band, green light Bg in the normal band, blue light Bb in the normal band, narrow-band blue light Nb, optical absorption characteristics W of plasma, and a peak wavelength Wp of the optical absorption coefficient of plasma.

As shown in FIG. 2, the optical absorption characteristics W of plasma are low around a wavelength of 415 nm, peak around a wavelength of 465 nm, and approach 0 around a wavelength of 550 nm.

Therefore, blue light Bb may be in the normal band, but in order to detect plasma in a significant manner, it may be particularly preferable that blue light Bb be narrowed in bandwidth so that the central wavelength thereof matches the peak wavelength Wp of the optical absorption coefficient of plasma. For example, blue light Bb is narrowed in bandwidth so that the central wavelength thereof is around a wavelength of 465 nm, and is used as narrow-band blue light Nb. Blue light Bb may also be narrowbanded to have a central wavelength of 460 nm to 470 nm. Furthermore, blue light Bb may be narrowbanded to have a central wavelength of 415 nm to 495 nm.

When irradiated with special light including red light Br and green light Bg as well as narrow-band blue light Nb, the plasma absorbs more blue light than red light and green light, resulting in a more pronounced yellowish appearance than when irradiated with normal light including standard blue light Bb.

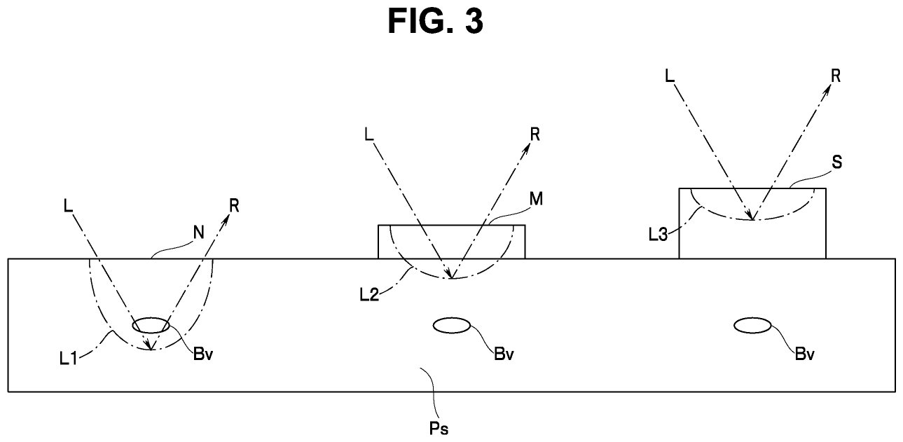

Next, FIG. 3 schematically shows a cross-section of the mucosa. FIG. 3 shows normal mucosa N, edema M, polyp S, blood vessel Bv, and the illumination light L. Here, the illumination light L is short-wavelength monochromatic light, such as narrow-band blue light Nb. The pigment in the mucosa is plasma.

As shown in a light penetration region L1, in the normal mucosa N, the illumination light L has a high penetration degree, and the reflected light R appears pale yellow due to the pigment in the mucosa having a higher optical absorption coefficient on a short-wavelength side than on a long-wavelength side.

As shown in a light penetration region L2, in the edema M, the penetration degree of the illumination light L is reduced compared to that in the normal mucosa N. More specifically, in the edema M, the illumination light L is scattered more on the short-wavelength side than on the long-wavelength side due to the thickened epithelium, and is reflected without being absorbed by the pigment in the mucosa. Therefore, in the edema M, the reflected light R appears more whitish than in the normal mucosa N.

As shown in a light penetration region L3, in the polyp S, the penetration degree of light is further reduced compared to that in the edema M, and the reflected light R appears even more whitish than in the edema M.

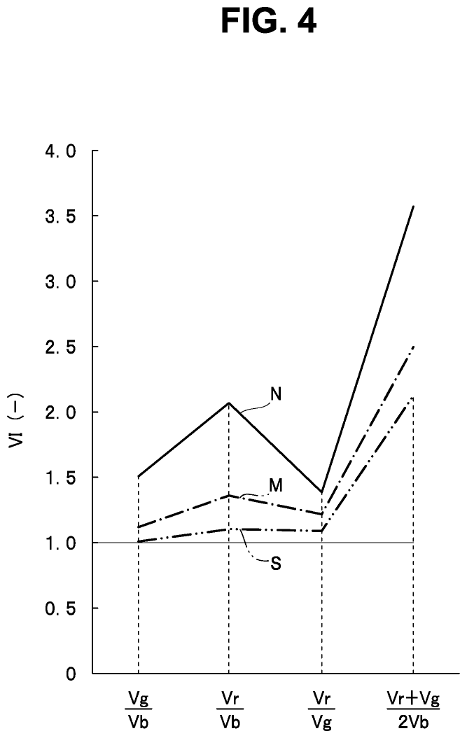

FIG. 4 shows an index VI where a green pixel value Vg, a red pixel value Vr, a blue pixel value Vb, or a sum of the green pixel value Vg and the red pixel value Vr of pixels included in the endoscopic image are each normalized. The pixel value V is acquired, for example, as 8-bit data (0 to 255).

FIG. 4 shows differences in the index VI among the normal mucosa N, the edema M, and the polyp S, based on different calculation formulas for the index VI. In FIG. 4, expressions “Vg/Vb”, “Vr/Vb”, “Vr/Vg”, and “(Vr+Vg)/2Vb” on the X-axis represent calculation formulas for the index VI, and the Y-axis represents the index VI normalized by each calculation formula.

The solid line indicates the normal mucosa N, the single-dot chain line indicates the edema M, and the double-dot chain line indicates the polyp S. Hereafter, the edema M and the polyp S will be referred to as the abnormal mucosa.

In mucosa in the body, such as the mucosa of the nasal sinuses, the symptom level becomes more severe in the order of the normal mucosa N, the edema M, and the polyp S. There is a color difference between the normal mucosa N and the abnormal mucosa, and as the symptom level becomes more severe, the mucosal epithelium thickens and the whiteness in appearance also becomes more pronounced. For this reason, the calculation formula yielding the largest index VI for the normal mucosa N and the largest index VI for the polyp S is “(Vr+Vg)/2Vb”.

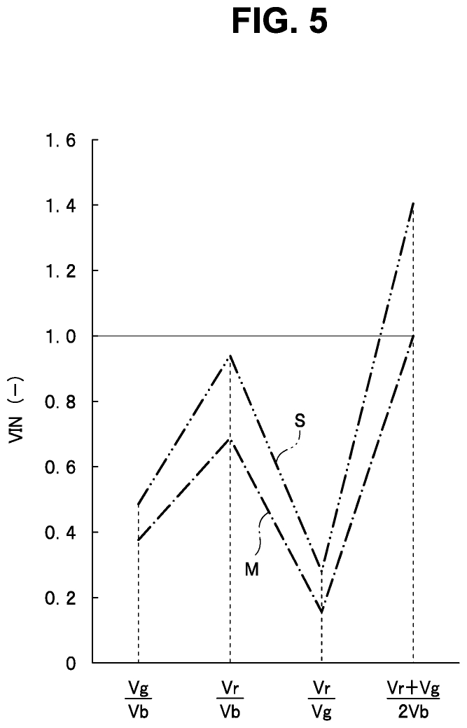

FIG. 5 shows indexes VIN for the edema M and the polyp S obtained by normalizing the same indexes VI for the edema M and the polyp S as in FIG. 4 by the index VI for the normal mucosa N. In FIG. 5, the X-axis shows calculation formulas used to calculate the indexes VIN normalized by the index VI for the normal mucosa N, and the Y-axis shows index VIN.

As shown in FIGS. 4 and 5, for the normal mucosa N and the polyp S, the indexes VI and VIN calculated using the calculation formula “(Vr+Vg)/2Vb” are larger than the indexes VI and VIN calculated using other index calculation formulas.

That is, the indexes VI and VIN calculated using the calculation formula “(Vr+Vg)/2Vb” strongly represent the difference between the color of the normal mucosa N and the color of the abnormal mucosa.

### Method of Operating Endoscope Apparatus

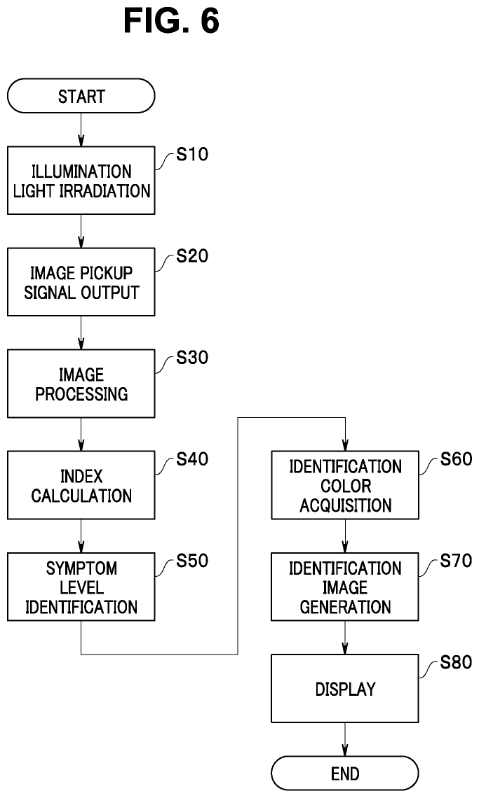

A method of operating the endoscope apparatus 1 will be described using the flowchart in FIG. 6.

### <Step S10> Illumination Light Irradiation

The insertion portion 11 of the endoscope 10 is inserted into the living body of the examinee 90, for example, into the nasal cavity. The illumination light L from the light source apparatus 20 is irradiated onto the mucosa, which is the object 91, via the illumination unit 16 at the distal end portion 11A. The illumination light L consists of red light Br, green light Bg, and blue light Bb.

### <Step S20> Image Pickup Signal Output

The image pickup unit 15 at the distal end portion 11A receives the reflected light R from the object 91, converts the light into an electrical signal, and outputs an image pickup signal to the processor 30.

### <Step S30> Image Processing

The image processing section 31 is an image processing circuit that performs, based on the image pickup signal, image processing such as, for example, gain adjustment, white balance adjustment, gamma correction, contour enhancement correction, and scaling adjustment, to generate an endoscopic image, which is an object image.

### <Step S40> Index Calculation

The calculation section 32 normalizes a sum of the red pixel value Vr and the green pixel value Vg of each of the plurality of pixels of the object image by twice the blue pixel value Vb (Nb), thereby calculating an index VI of each pixel. In other words, the calculation section 32 calculates indexes VI of a plurality of regions (pixels) of the object.

In FIGS. 4 and 5, an expression “(Vr+Vg)/2Vb” is used as a calculation formula that normalizes pixel values and calculates the indexes VI. However, as long as the calculation formula normalizes a sum of the red pixel value and the green pixel value by twice the blue pixel value, the formula can be altered as appropriate.

For example, the index VI may be converted into 8-bit (0 to 255) data, a numerical value may be further added to the 8-bit data, or the k value in a calculation formula “(Vr+Vg)/kVb” may be modified. In the following, the index VI is calculated using formula 1.

VI=32*log2[(Vr+Vg)/2Vb]+256 <Formula 1>

The calculation section 32 may preferably calculate the index VI using one of a plurality of calculation formulas corresponding respectively to a plurality of objects 91 (for example, the nasal sinuses and the digestive tract).

### <Step S50>Symptom Level Identification

The identification section 33 is an identification circuit that identifies a symptom level of each of the pixels, based on the index VI, by using a plurality of thresholds T.

In the endoscope apparatus 1, the symptom level includes five types: “normal,” “mild,” “moderate,” “severe,” and “most severe.” When there are three or more types of symptom levels, more detailed determination of symptoms is facilitated than when there are two types (“normal” and “abnormal”).

In order to identify the five types of symptom levels, four thresholds T (a first threshold T1 to distinguish between normal and mild levels, a second threshold T2 to distinguish between mild and moderate levels, a third threshold T3 to distinguish between moderate and severe levels, and a fourth threshold T4 to distinguish between severe and most severe levels) are required. The threshold T is set in advance as appropriate based on determinations by a plurality of experts. It is needless to say that the relationship of magnitudes of the four thresholds T, each set in a predetermined range, is T1 to T4.

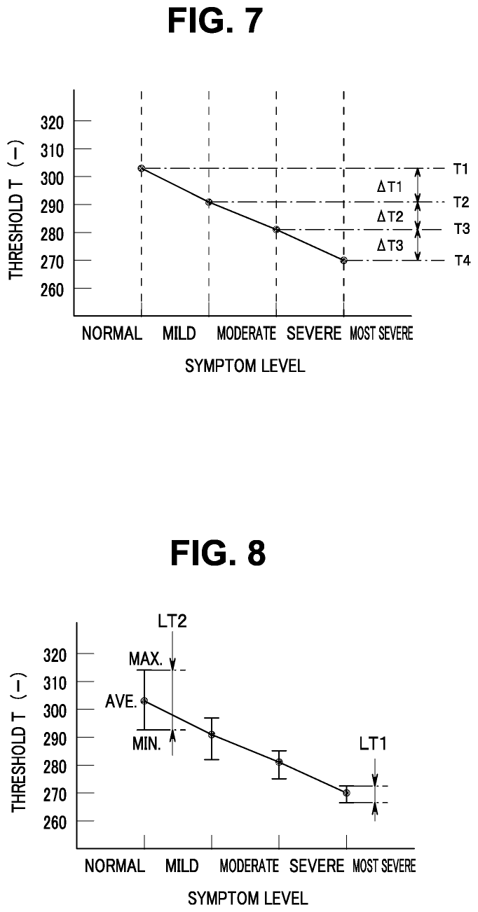

FIG. 7 shows an example of the thresholds T. It may be preferable that differences ΔT between the plurality of thresholds are substantially the same. In other words, the plurality of thresholds may be set at substantially equal intervals. For example, it may be preferable that a difference ΔT3 (threshold T4 minus threshold T3) is greater than or equal to 80% and less than or equal to 120% of a difference ΔT2 (threshold T3 minus threshold T2).

However, depending on conditions, a first threshold difference ΔT1 between the first threshold T1 and the second threshold T2 may be greater than a second threshold difference ΔT2 between the second threshold T2 and the third threshold T3, and the second threshold difference ΔT2 may be greater than a third threshold difference ΔT3 between the third threshold T3 and the fourth threshold T4. Conversely, the first threshold difference ΔT1 may be smaller than the second threshold difference ΔT2, and the second threshold difference ΔT2 may be smaller than the third threshold difference ΔT3.

Depending on a combined system, the image pickup device mounted, or the like, the threshold difference ΔT may in some cases become larger as the severity degree rises.

It should be noted that the memory 50 stores a plurality of threshold sets, which correspond respectively to the plurality of objects 91 and each include a plurality of thresholds, and the identification section 33 performs identification using a threshold set corresponding to the object 91. The threshold set used by the identification section 33 may be acquired automatically or set with the setting section 36.

As shown in FIG. 8, each of the thresholds T (T1 to T4) included in the plurality of threshold sets has a variation (dispersion), but it may be preferable that the variation of each of the thresholds T (T1 to T4) is set in a predetermined range. For example, the maximum value of the threshold T1 is 313, and the minimum value of the threshold T1 is 291. In contrast, the maximum value of the threshold T4 is 273, and its minimum value is 267. That is, a variation LT1 (=6) of the threshold T4 is smaller than a variation LT2 (=22) of the threshold T1.

This is caused by color balance calibration performed by the image processing section 31. That is, this is because the image processing section 31 performs white balance as color balance calibration, with reference to white color close to the color of the most severe region.

It is possible to adjust the variations of the thresholds T by altering the color used for color balance calibration or by performing calibration using a plurality of colors. For example, by performing color balance calibration using a color of the normal region (red to yellow), the variation LT2 of the threshold T1 can be made small. By performing color balance calibration using an intermediate color between the color of the most severe region and the color of the normal region (red to yellow), the variations of the plurality of thresholds T can be made small and averaged.

### <Step S60> Identification Color Acquisition Section

The identification color acquisition section 34 acquires an identification color in accordance with the symptom level of a pixel acquired by the identification section 33.

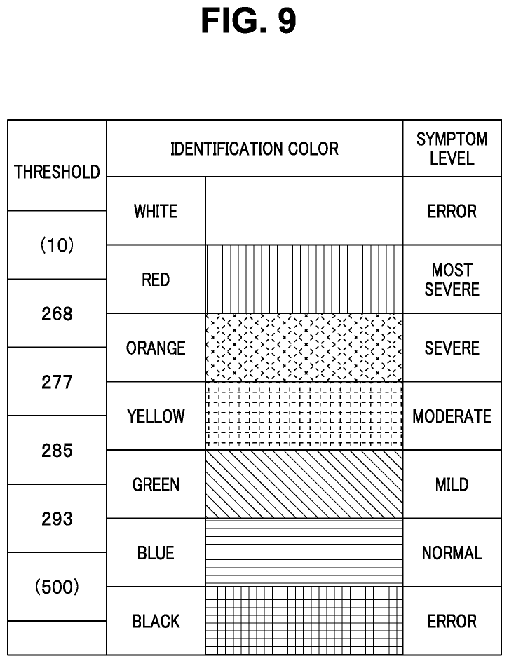

FIG. 9 shows identification colors in accordance with the symptom levels. The index VI is data in a range (0 to 511), obtained, for example, by adding 256 to 8-bit data. The plurality of thresholds T and the identification colors are stored in the memory 50.

It should be noted that, in the example of FIG. 9, the identification color acquisition section 34 acquires a plurality of colors with different hues, but may also acquire a plurality of saturations with different vividness, a plurality of lightnesses with different brightnesses, a plurality of hatchings with different intervals, a plurality of designs with different patterns, or the like.

It should be noted that an endoscopic image may include a pixel having an erroneous pixel value of a color that is not produced in normal imaging. In the endoscope apparatus 1, a pixel having a pixel value V, wherein at least one of the red pixel value, the green pixel value, or the blue pixel value is less than or equal to a predetermined lower limit pixel value or greater than or equal to a predetermined upper limit pixel value, is defined as a first error pixel. For example, a pixel whose pixel value V, which is in a range of 0 to 255, is less than or equal to 5 or greater than or equal to 250 is the first error pixel.

Furthermore, a pixel whose index VI is less than or equal to a predetermined lower limit threshold or greater than or equal to a predetermined upper limit threshold is defined as a second error pixel. For example, in the example shown in FIG. 9, a pixel whose index VI is less than or equal to a lower limit threshold 10 or greater than or equal to an upper limit threshold 500 is the second error pixel.

The identification color acquisition section 34 acquires an error color for the error pixels (the first error pixel and the second error pixel). The image generation section generates an identification image using the error color for the error pixels. The error color is not limited to white or black as illustrated in FIG. 9, but may also be gray or the like, for example. Pixels whose threshold or the like is less than or equal to a lower limit and greater than or equal to an upper limit may have the same error color (for example, white). The numerical values serving as determination criteria for error pixels and error color data are stored in the memory 50.

It should be noted that a ratio (error ratio) of the number of error pixels to the total number of pixels may be calculated, and if the error ratio is greater than or equal to a predetermined value, the processor 30 may produce a warning. The number of error pixels is one of the number of first error pixels, the number of second error pixels, or the total number of the error pixels. The warning is displayed on the monitor 40 in text or graphics, for example.

By, for example, altering the threshold set based on the warning, it becomes easier to identify the symptom level. It should be noted that the threshold set may be altered automatically or by the user.

### <Step S70> Identification Image Generation

The identification color acquisition section 34 acquires an identification color corresponding to the symptom level of each pixel. The image generation section 35 includes an identification color acquisition circuit configured to generate an identification image using respective identification colors of a plurality of pixels.

### <Step S80> Display

The monitor 40 displays the identification image in various forms.

### Display Example 1

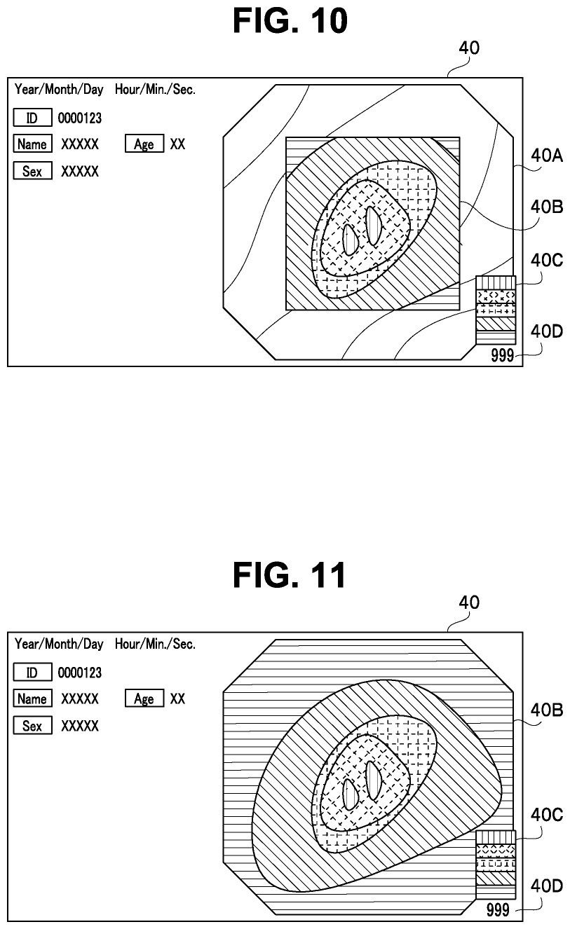

FIG. 10 shows an example of an image displayed on the monitor 40. In FIG. 10, a partial region of the endoscopic image 40A displayed in color is displayed replaced with an identification image 40B. In other words, a superimposed image, in which the identification image 40B is superimposed on the endoscopic image 40A, is displayed. On the endoscopic image 40A, a range displayed as the identification image 40B is indicated by a frame.

From the perspective of visibility and operability, it may be preferable that the area of the identification image 40B be greater than or equal to 20% and less than or equal to 70% of the area of the entire region of the endoscopic image 40A. The area of the identification image 40B can be altered, for example, by operation of the setting section 36.

The monitor 40 displays an identification color list display 40C as well as an average index value 40D. That is, the calculation section 32 calculates the average index value 40D of a plurality of pixels, and the monitor 40 displays the average index value 40D.

Based on the average index value 40D, the user can easily grasp the symptom of the examinee.

### Display Example 2

As shown in FIG. 11, the monitor 40 displays the identification image 40B instead of the endoscopic image 40A. That is, the calculation section 32 may calculate an index from a plurality of pixels of the entire region of the endoscopic image 40A, which is the object image, and the image generation section 35 may generate an identification image corresponding to the entire region of the endoscopic image 40A.

### Display Example 3

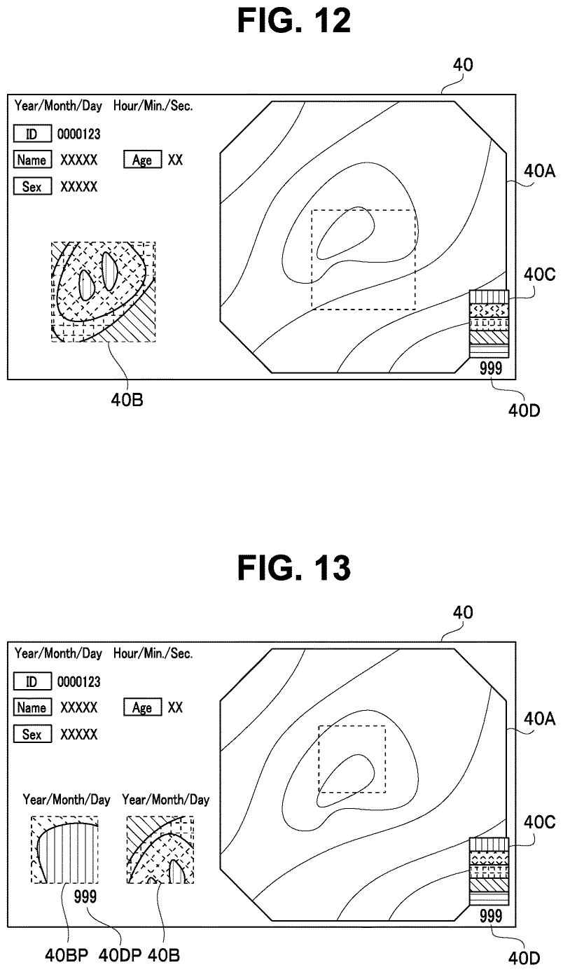

As shown in FIG. 12, on the monitor 40, the identification image 40B in a region enclosed by a frame in the endoscopic image 40A is displayed in a region separate from the endoscopic image 40A. At least one of the position or range (area) of a partial region that generates the identification image 40B in the entire region of the endoscopic image 40A, which is the object image, can be selected as appropriate by operation of the setting section 36.

In a narrow conduit, it may not be easy to direct the center of the endoscopic image 40A (the center of the field of view of the image pickup unit 15) toward a region of interest. However, by selecting at least one of the position or range of the region in which to display the identification image 40B, the user can easily identify the region of interest.

### Display Example 4

As shown in FIG. 13, the monitor 40 displays, together with the endoscopic image 40A and the identification image 40B, an identification image 40BP, which is a past image obtained in a range similar to that of the identification image 40B at a past examination of the examinee 90, and an average index value 40DP.

Selection of the range of the past identification image 40BP, based on the identification image 40B, is performed using pattern matching of the endoscopic image or the identification image, the state at the time of the examination (for example, the position and direction of the distal end portion of the endoscope), and the like.

A display screen showing the endoscopic image 40A and the identification image 40B, which are obtained at the time of the examination, and a display screen showing the endoscopic image and the identification image 40BP, which are obtained in the past, may be displayed switchably or displayed on separate monitors. It may be preferable that the display range and the display position of the past identification image 40BP can also be altered as appropriate. The identification image 40BP and the like obtained at a past examination of the examinee 90 are stored in, for example, a server in the hospital, and transferred to the processor 30.

It should be noted that a difference between the past identification image 40BP and the identification image 40B obtained at the time of the examination may be calculated, and the difference may be illustrated. For example, by displaying only pixels in which the difference in the index VI of each pixel has increased by a predetermined value or more, regions where the disease state has changed can be clearly indicated.

### Display Example 5

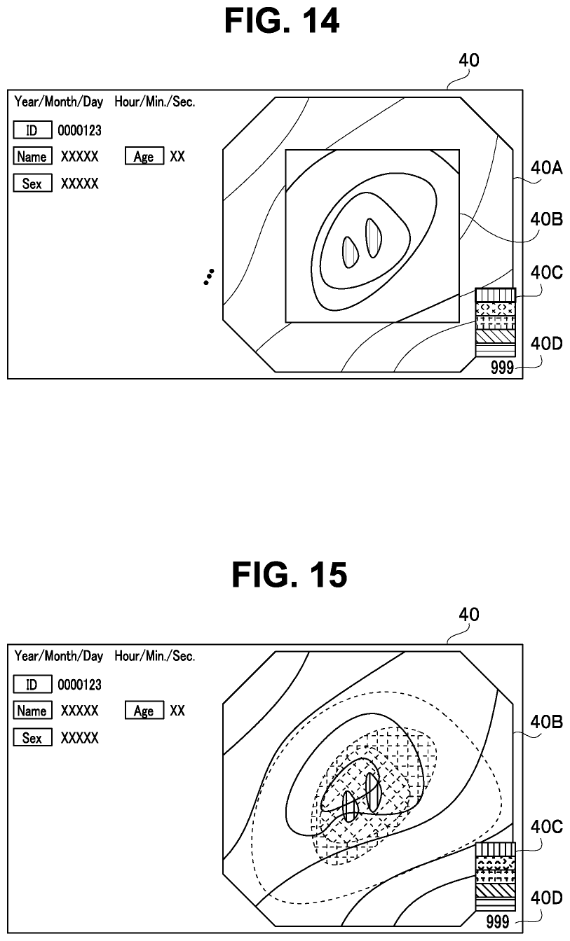

The screen of the monitor 40 in FIG. 14 is similar to that in FIG. 10, but in the identification image 40B, only red at the most severe level among the five color regions is displayed in color, and regions from the normal level to the severe level are displayed in monochrome white or gray. Alternatively, the regions from the normal level to the severe level may be displayed as a standard endoscopic image illuminated with white light.

The region of the level (color) displayed in color may be set in advance, but may preferably be adjustable as appropriate. For example, performing a click operation once on each of the five color regions in the identification color list display 40C causes that level region to be changed from being displayed in color in the identification color to being displayed in white, and performing a click operation again causes the level region to be changed from being displayed in white to being displayed in color. A plurality of level regions may be displayed in color. A standard endoscopic image illuminated with white light may be displayed in the region of the identification image that is displayed in white.

### Display Example 6

The screen of the monitor 40 in FIG. 15 is similar to that in FIG. 10, but differs from FIG. 10 in that the identification image 40B is rendered semi-transparent by setting its transmittance, and is displayed superimposed on the endoscopic image 40A. At 0% transmittance, only the identification image is displayed and the endoscopic image 40A is not displayed, and at 100%, only the endoscopic image 40A is displayed and the identification image 40B is not displayed.

Furthermore, the transmittance of each of the identification colors of the five color regions of the identification image 40B can be set for each of the identification colors. Of course, only a selected identification color may be rendered semi-transparent.

The semitransparency setting for the identification image 40B is performed, for example, through the monitor 40 serving as a touch panel. When each of the five color regions in the identification color list display 40C is long-pressed and slid to the left, the transmittance of the identification color of that level region increases, and when slid to the right, the transmittance of the identification color decreases.

TABLE 1

| | Transmittance (%) | | |
|---|---|---|---|
| Level (Color) | A | B | C |
| Most severe (Red) | 50 | 50 | 50 |
| Severe (Orange) | 50 | 40 | 0 |
| Moderate (Yellow) | 50 | 30 | 0 |
| Mild (Green) | 50 | 0 | 0 |
| Normal (Blue) | 50 | 0 | 0 |

In Case A of Table 1 above, all identification colors (levels) are set to 50% transmittance.

In Case B of Table 1 above, the transmittance becomes smaller as a change is made from the most severe level to the moderate level, and is set to 0% transmittance for the mild level and the normal level. FIG. 15 shows a screen of the monitor 40 for Case B.

In Case C of Table 1 above, only the most severe level has 50% transmittance, and the other levels are set to 0% transmittance.

It should be noted that, if there is a region set to 100% transmittance, only the endoscopic image 40A is displayed in that region, making it impossible to recognize on which part of the image the identification image 40B is displayed. For this reason, the display may be made as shown in Cases 1 to 3 below.

(Case 1) As shown in FIG. 14, when the identification image 40B, in which there is a region with 100% transmittance, is superimposed only on the central part of the endoscopic image 40A, the boundary of the identification image 40B is displayed as a solid line or a dashed line.

(Case 2) Although not shown, when only the most severe (red) part has 50% transmittance, and the other levels have 100% transmittance, the boundary between the most severe region and the other regions is displayed as a solid line or a dashed line.

(Case 3) When a plurality of levels are set to 100% transmittance, such as when the levels from the moderate level to the normal level are set to 100% transmittance, the boundaries between the plurality of level regions may not be recognizable on the screen. Although not shown, the boundaries between the plurality of level regions are displayed as a solid line or a dashed line. For example, by displaying, as solid lines, the boundary between the moderate severity region and the mild severity region and the boundary between the mild severity region and the normal region, the boundaries between the regions are recognized even when a plurality of levels have 100% transmittance.

### Display Example 7

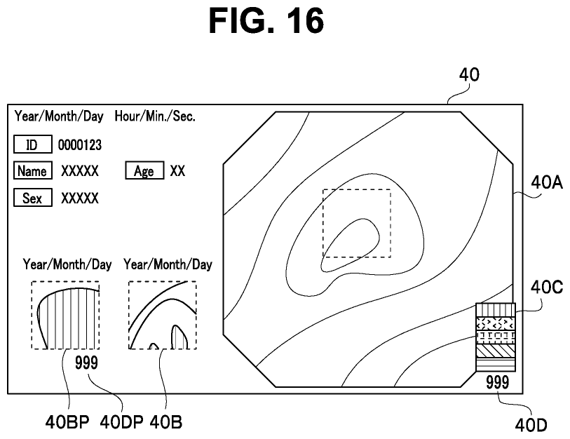

The screen of the monitor 40 in FIG. 16 is similar to that in FIG. 13, and displays, together with the endoscopic image 40A and the identification image 40B, the identification image 40BP obtained in a range similar to that of the identification image 40B at a past examination of the examinee 90. However, unlike FIG. 13, in the identification image 40B, only red which is the most severe level among the five color regions is displayed in color, and regions from the normal level to the severe level are displayed in white.

The most severe level is a level having the largest number of pixels, or in other words, the largest area, in the past identification image 40BP. The level displayed in color on the identification image 40B is not limited to the most severe level, and can be set as appropriate.

As described above, a method of operating an endoscope system of another embodiment involves that an image pickup unit 15 of an endoscope 10 picks up an image of an object 91 in the body of an examinee 90 and outputs an image signal, the processor 30 performs image processing on the image signal to generate an object image, normalizes a sum of a red pixel value and a green pixel value of each of a plurality of pixels in at least a partial region of the object image by twice a blue pixel value to thereby calculate an index of each of the pixels, stores a plurality of thresholds for identifying a plurality of regions of the object as one of three or more types of symptom levels and identification colors corresponding respectively to the plurality of symptom levels, identifies a symptom level of each of the pixels based on the index by using the plurality of thresholds, acquires the identification color corresponding to the symptom level of each of the pixels, generates an identification image using the respective identification colors of the plurality of pixels, and the monitor 40 displays the identification image.

A program of another embodiment causes a computer to execute the above processing.

A non-transitory computer-readable storage medium of another embodiment stores a program that causes a computer to perform the above processing.

It should be noted that the above-described numerical ranges, such as wavelength, are not limited to the above-described ranges, and can be increased or decreased as appropriate. The endoscope 10 may be a rigid endoscope in which the insertion portion 11 is rigid. The present disclosure is not limited to the above-mentioned embodiments, etc., and various alterations, modifications, etc. can be made without departing from the gist of the present disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, or other storage media) that stores data, software, or instructions contributing to the processing operations. Accordingly, the processing circuitry may be implemented as a specialized hardware circuit having fixed logic, a programmable circuit executing instructions stored in an internal or external memory, or any combination thereof. Furthermore, like the “processor” described above, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope processor comprising one or more processors configured to:
receive an image signal to generate an object image;
normalize, by a blue pixel value, a sum of a red pixel value and a green pixel value of at least one pixel of the object image to thereby calculate an index of the pixel;
perform identification using a plurality of thresholds for performing, based on the index, identification as one of a plurality of types of symptom levels;
acquire an identification color corresponding to each of the symptom levels; and
generate an identification image using the identification color of the pixel.

Clause 2: The endoscope processor according to clause 1, wherein the plurality of types of symptom levels include normal, mild, moderate, severe, and most severe levels.

Clause 3: The endoscope processor according to clause 1, wherein each of the plurality of thresholds is set in a predetermined range.

Clause 4: The endoscope processor according to clause 1, wherein differences between the plurality of thresholds are the same.

Clause 5: The endoscope processor according to clause 2, wherein
a first threshold difference between a first threshold, which distinguishes between the normal level and the mild level, and a second threshold, which distinguishes between the mild level and the moderate level, is greater than a second threshold difference between the second threshold and a third threshold, which distinguishes between the moderate level and the severe level, and the second threshold difference is greater than a third threshold difference between the third threshold and a fourth threshold, which distinguishes between the severe level and the most severe level.

Clause 6: The endoscope processor according to clause 2, wherein
a first threshold difference between a first threshold, which distinguishes between the normal level and the mild level, and a second threshold, which distinguishes between the mild level and the moderate level, is smaller than a second threshold difference between the second threshold and a third threshold, which distinguishes between the moderate level and the severe level, and the second threshold difference is smaller than a third threshold difference between the third threshold and a fourth threshold, which distinguishes between the severe level and the most severe level.

Clause 7: The endoscope processor according to clause 2, wherein at least one of the one or more processors is configured to acquire a plurality of threshold sets corresponding respectively to a plurality of objects, each threshold set including the plurality of thresholds.

Clause 8: The endoscope processor according to clause 7, wherein variation in the thresholds to distinguish between the severe level and the most severe level among the plurality of thresholds in the plurality of threshold sets is smaller than variation in the thresholds to distinguish between the normal level and the mild level.

Clause 9: The endoscope processor according to clause 8, wherein at least one of the one or more processors is configured to perform white balance adjustment using white color as color balance calibration.

Clause 10: The endoscope processor according to clause 7, wherein variation in the thresholds is controlled by a color used for color balance calibration.

Clause 11: The endoscope processor according to clause 10, wherein at least one of the one or more processors is configured to perform, as the color balance calibration, adjustment using an intermediate color between a color of a normal region and a color of a most severe region.

Clause 12: The endoscope processor according to clause 1, wherein at least one of the one or more processors is configured to calculate the index using one of a plurality of calculation formulas corresponding respectively to a plurality of objects.

Clause 13: The endoscope processor according to clause 1, wherein
an identification color received by the one or more processors includes an error color as the identification color for each of a first error pixel and a second error pixel, the first error pixel having a pixel value wherein at least one of the red pixel value, the green pixel value, or the blue pixel value is less than or equal to a predetermined lower limit pixel value or greater than or equal to a predetermined upper limit pixel value, and the second error pixel having an index that is less than or equal to a predetermined lower limit threshold or is greater than or equal to a predetermined upper limit threshold, and
the processor is configured to generate the identification image using the error color for the first error pixel and the second error pixel.

Clause 14: The endoscope processor according to clause 13, wherein a warning is outputted when a ratio of the number of the first error pixels, the number of the second error pixels, or a sum of the number of the first error pixels and the number of the second error pixels to a total number of pixels is greater than a predetermined ratio.

Clause 15: The endoscope processor according to clause 1, wherein at least one of the one or more processors is configured to output, together with the identification image, a past image that is an identification image generated based on a past object image captured of a same object.

Clause 16: The endoscope processor according to clause 15, wherein the past image is displayed such that an identification color is selected from respective identification colors of the at least one pixel, only pixels of the selected identification color are displayed as an identification image, and other pixels are displayed in white or as an endoscope image.

Clause 17: The endoscope processor according to clause 1, wherein at least one identification color is selected from the respective identification colors of the at least one pixel, only pixels of the selected identification color are displayed in color, and other pixels are displayed in white.

Clause 18: The endoscope processor according to clause 1, wherein the identification image is rendered semi-transparent and displayed on a monitor in a manner superimposed on the object image.

Clause 19: The endoscope processor according to clause 17, wherein the identification image is rendered semi-transparent with a different transmittance for each of a plurality of identification colors.

Clause 20: The endoscope processor according to clause 17, wherein only an identification color selected from a plurality of identification colors is rendered semi-transparent.

Clause21: An endoscope system comprising:
the endoscope processor according to clause 1;
an endoscope connected to the endoscope processor, the endoscope being configured to output the image signal;
a memory connected to the endoscope processor, the memory being configured to store the threshold and the identification color; and
a monitor connected to the endoscope processor.

What is claimed is:

1. An endoscope processor comprising:
a connection interface; and
processing circuitry configured to:
receive, via the connection interface, an image signal to generate an object image;
normalize, by a blue pixel value, a sum of a red pixel value and a green pixel value of at least one pixel of the object image, thereby calculating an index of the at least one pixel;
based on the index, identify which of a plurality of symptom levels defined using a plurality of thresholds the at least one pixel falls into;
acquire an identification color corresponding to the identified symptom level; and
generate an identification image using the identification color of the at least one pixel.

2. The endoscope processor according to claim 1, wherein the plurality of types of symptom levels include normal, mild, moderate, severe, and most severe levels.

3. The endoscope processor according to claim 1, wherein each of the plurality of thresholds is set within a predetermined range.

4. The endoscope processor according to claim 1, wherein the plurality of thresholds are set at substantially equal intervals.

5. The endoscope processor according to claim 2, wherein
the plurality of thresholds include:
a first threshold that distinguishes between the normal level and the mild level;
a second threshold that distinguishes between the mild level and the moderate level;
a third threshold that distinguishes between the moderate level and the severe level; and
a fourth threshold that distinguishes between the severe level and the most severe level,
a first threshold difference between the first threshold and the second threshold is greater than a second threshold difference between the second threshold and the third threshold, and
the second threshold difference is greater than the third threshold difference between the third threshold and the fourth threshold.

6. The endoscope processor according to claim 2, wherein
the plurality of thresholds include:
a first threshold that distinguishes between the normal level and the mild level;
a second threshold that distinguishes between the mild level and the moderate level;
a third threshold that distinguishes between the moderate level and the severe level; and
a fourth threshold that distinguishes between the severe level and the most severe level,
a first threshold difference between the first threshold and the second threshold is smaller than a second threshold difference between the second threshold and the third threshold, and
the second threshold difference is smaller than a third threshold difference between the third threshold and the fourth threshold.

7. The endoscope processor according to claim 2, wherein the processing circuitry is configured to acquire a plurality of threshold sets respectively corresponding to a plurality of objects, each threshold set including a respective plurality of thresholds.

8. The endoscope processor according to claim 7, wherein a variation in a threshold that distinguishes between the severe level and the most severe level among the respective plurality of thresholds in each threshold set is smaller than a variation in a threshold that distinguishes between the normal level and the mild level.

9. The endoscope processor according to claim 8, wherein the processing circuitry is configured to perform white balance adjustment using white as color balance calibration.

10. The endoscope processor according to claim 7, wherein a variation in at least one threshold within the plurality of threshold sets is controlled by a color used for color balance calibration.

11. The endoscope processor according to claim 10, wherein the processing circuitry is configured to perform, as the color balance calibration, adjustment using an intermediate color between a color of a normal region and a color of a most severe region.

12. The endoscope processor according to claim 1, wherein the processing circuitry is configured to calculate the index using one of a plurality of calculation formulas, each corresponding to a respective one of a plurality of objects.

13. The endoscope processor according to claim 1, wherein
the processing circuitry is configured to:
acquire a plurality of identification colors including:
a first error color as the identification color for a first error pixel, the first error pixel being a pixel in which at least one of the red pixel value, the green pixel value, or the blue pixel value is less than or equal to a predetermined lower limit pixel value or greater than or equal to a predetermined upper limit pixel value; and/or
a second error color as the identification color for a second error pixel, the second error pixel being a pixel whose index is less than or equal to a predetermined lower limit threshold or greater than or equal to a predetermined upper limit threshold; and
generate the identification image using the first error color and/or the second error color.

14. The endoscope processor according to claim 13, wherein the processing circuitry is configured to output a warning when a ratio of a count of the first error pixels, a count of the second error pixels, or a sum of the counts of the first and second error pixels to a total count of pixels is greater than a predetermined ratio.

15. The endoscope processor according to claim 1, wherein the processing circuitry is configured to output a past image together with the identification image, the past image being an identification image generated based on a past object image captured of the same object.

16. The endoscope processor according to claim 15, wherein
the outputting of the past image comprises:
selecting an identification color from one or more identification colors, each corresponding to a respective one of the at least one pixel;
displaying only pixels of the selected identification color as an identification image; and
displaying other pixels in white or as an endoscope image.

17. The endoscope processor according to claim 1, wherein
the processing circuitry is configured to:
select at least one identification color from one or more identification colors, each corresponding to a respective one of the at least one pixel;
display only pixels of the selected identification color in color; and
display other pixels in white.

18. The endoscope processor according to claim 1, wherein the identification image is rendered semi-transparent and superimposed on the object image.

19. The endoscope processor according to claim 17, wherein the identification image is rendered semi-transparent with different transmittances respectively for a plurality of identification colors.

20. The endoscope processor according to claim 17, wherein only the selected at least one identification color is rendered semi-transparent.

21. An endoscope system comprising:
an endoscope processor comprising:
a connection interface; and
processing circuitry configured to:
receive, via the connection interface, an image signal to generate an object image;
normalize, by a blue pixel value, a sum of a red pixel value and a green pixel value of at least one pixel of the object image, thereby calculating an index of the at least one pixel;
based on the index, identify which of a plurality of symptom levels defined using a plurality of thresholds the at least one pixel falls into;
acquire an identification color corresponding to the identified symptom level; and
generate an identification image using the identification color of the at least one pixel;
an endoscope connected to the endoscope processor via the connection interface, the endoscope being configured to output the image signal;
a memory connected to the endoscope processor, the memory being configured to store the thresholds and identification color data; and
a monitor connected to the endoscope processor.
